(19) United States
(12) Patent Application Publication
(10) Pub. No.: US 2021/0213017 A1
(43) Pub. Date: Jul. 15, 2021

(54) METHOD OF TREATMENT AND CLINICAL TRIAL DESIGN FOR GEOGRAPHIC ATROPHY DUE TO AGE-RELATED MACULAR DEGENERATION

(71) Applicant: Allergan, Inc., Irvine, CA (US)

(72) Inventors: Kevin Kerr, Laguna Niguel, CA (US); Francisco Lopez, Ladera Ranch, CA (US)

(21) Appl. No.: 17/103,613

(22) Filed: Nov. 24, 2020

Related U.S. Application Data
(63) Continuation of application No. 15/971,555, filed on May 4, 2018, now abandoned.
(60) Provisional application No. 62/502,375, filed on May 5, 2017.

(51) Int. Cl. A61K 31/517; A61P 27/02; A61K 49/00; A61K 47/34; A61K 9/00; A61F 9/00; A61K 31/498

(52) U.S. Cl. CPC A61K 31/517; A61P 27/02; A61K 49/0004; A61K 31/498; A61K 9/0051; A61F 9/0017; A61K 47/34

(57) ABSTRACT

Methods of treating or slowing the growth of a lesion associated with geographic atrophy and methods of evaluating a drug or agent for use in treating, reducing the progression of or slowing the growth of a lesion associated with geographic atrophy.

### CROSS-REFERENCE TO RELATED APPLICATIONS

This application is a continuation of U.S. patent application Ser. No. 15/971,555, filed May 4, 2018, which claims the benefit of U.S. Provisional Application No. 62/502,375, filed on May 5, 2017, the entire contents of each application is incorporated herein by reference.

### TECHNICAL FIELD

The subject matter described herein relates to methods of treating or slowing the growth of a lesion associated with geographic atrophy as well as methods of evaluating a drug or agent for use in treating, reducing the progression of or slowing the growth of a lesion associated with geographic atrophy, especially on a reduced time scale.

### BACKGROUND

Age-related macular degeneration (AMD) is a retinal disease that is the primary cause of blindness and visual disability for adults over the age of 60 in the developed world (Mata and Vogel, Curr Opin Ophthalmol, 21:190-196, 2010). AMD is generally categorized as two principle types, non-exudative or exudative AMD. Non-exudative AMD (also non-neovascular or dry AMD) is characterized by atrophy of the layers of the macula (including the photoreceptors and retinal pigment epithelium). Small drusen (yellow deposits formed of lipids and proteins) may appear under the retina. The pathology of non-exudative AMD is characterized by thinning of the photoreceptor layer of the retina, variable atrophy and other changes of the RPE, thickening of Bruch's membrane, drusen formation, and decreased density of the choriocapillaris layer (Danis et al., Clin Ophthalmol, 9:2159-2174, 2015). Geographic atrophy (GA) accounts for 35% of late-stage or advanced non-exudative AMD. GA is a progressive form of non-exudative (dry) AMD that is characterized by irreversible loss of macular retinal tissue, retinal pigment epithelium (RPE), and/or choriocapillaris, e.g. that are non-functioning or atrophied. GA is responsible for severe vision loss in approximately 20% of all patients with AMD, and more than 8 million people are affected worldwide (Khan et al., ISRN Ophthalmology, 2014, 2014:608390). The RPE is essential for vision as it transports nutrients and ions, secretes growth factors and protects against photooxidation (Enslow et al., Ophthalmol Eve Dis, 8:31-32, 2016). GA may typically be defined as an area of atrophy of 175 μm or more with sharply demarcated borders. In GA patients, visual acuity (VA) can still be good if the macula is spared, but decreased if GA extends through the fovea causing a great impairment of quality of life. There are no approved treatments for non-exudative AMD or geographic atrophy as of yet. About 10% of all AMD cases progress to exudative AMD (also neovascular or wet AMD), which is characterized by neovascularization of unstable blood vessels in the choroid layer behind the retina (choroidal neovascularization or CNV). These new blood vessels may leak into the layers of the retina (e.g. macula) and resulting in vision loss. Exudative AMD may be treated by laser photocoagulation or anti-VEGF drugs to stop leaking of the new vessels.

Although a significant amount of research has been focused on treatments for non-exudative AMD, research has been hampered by the multifactorial nature of non-exudative AMD, its complex physiopathology, the lack of an animal model for non-exudative AMD, and the lack of in vitro systems for testing new drugs (Damico et al., Arg Bras Oftalmol, 75 (1):71-76, 2012).

It would be beneficial to provide a treatment for advanced non-exudative AMD or GA. It would also be beneficial to provide a system or method for providing a clinical assessment of potential treatments for treating patients with AMD including geographic atrophy, especially on a reduced time scale.

The foregoing examples of the related art and limitations related therewith are intended to be illustrative and not exclusive. Other limitations of the related art will become apparent to those of skill in the art upon a reading of the specification and a study of the drawings.

### SUMMARY

The following aspects and embodiments thereof described and illustrated below are meant to be exemplary and illustrative, not limiting in scope.

In first aspect a treatment method for slowing the rate of growth of a lesion associated with geographic atrophy is provided. In embodiments, the method comprises administering a composition comprising a therapeutically effective amount of a drug or therapeutic agent to a subject or patient in order to slow the rate of growth of a lesion associated with geographic atrophy. The therapeutically effective amount slows the rate of lesion growth or progression in a reduced time period such as 12 months in a population of subjects with a baseline geographic atrophy lesion area of greater than or equal to 8 mm² or a baseline effective diameter of greater than or equal to 3.19 mm relative to placebo-treated subjects in a population of subjects with a baseline geographic atrophy lesion area of greater than or equal to 8 mm² or a baseline effective diameter of greater than or equal to 3.19 mm.

In one embodiment, the drug is brimonidine or a salt thereof. In some embodiments, brimonidine may be brimonidine free base or a brimonidine salt such as brimonidine tartrate. In some embodiments, the composition comprises between about 25-60 wt % drug or therapeutic agent.

In some embodiments, the composition is an ocular implant. In some embodiments the ocular implant is a solid ocular implant. In some embodiments, the solid ocular implant is comprised of one or more biodegradable polymers. In some embodiments, at least one of the biodegradable polymers is poly (D,L-lactide) and/or a poly (D,L-lactide-co-glycolide) polymer. In some embodiments, the solid intraocular implant is comprised of between about 50-65 wt % of one or more biodegradable polymers.

In a second aspect, a method of slowing the progression of lesion size associated with geographic atrophy is provided. In embodiments, the method comprises administering to a subject or patient a drug or therapeutic agent that slows the progression of lesion size associated with geographic atrophy. In embodiments, the therapeutically effective dose of the drug is established by dosing a population of subjects with a baseline geographic atrophy lesion area of greater than or equal to about 8 mm² or a baseline effective diameter of greater than or equal to 3.19 mm. In embodiments, the method is effective to slow the progression of lesion size associated with geographic atrophy within about twelve months. In embodiments, the drug and composition are as described above.

In a third aspect a method to evaluate a drug for use in reducing progression of geographic atrophy in a subject is provided. In embodiments, the method comprises selecting a subject having a baseline geographic atrophy lesion area of greater than or equal to 8 mm² or a baseline effective diameter of greater than or equal to 3.19 mm for treatment with the drug, administering the drug to the subject, determining geographic atrophy lesion area or effective diameter, and repeating said administering and said determining n times, where n is at least 1. In embodiments, the drug is effective to reduce progression of geographic atrophy if the change in lesion area determined after the repeating step from the baseline geographic atrophy lesion area is less than a reference subject with a baseline geographic atrophy lesion area of greater than or equal to 8 mm² treated n times with a placebo or sham treatment. In other embodiments, the drug is effective to reduce progression of geographic atrophy if the change in lesion area determined after the repeating step from the baseline lesion effective diameter is less than a reference subject with a baseline lesion effective diameter of greater than or equal to 3.19 mm treated n times with a placebo or sham treatment. In embodiments, effects can be observed at or before about month 12. In embodiments, the drug and composition are as described above.

In embodiments, the determining step comprises transforming the geographic atrophy (GA) lesion area to an effective diameter (ED) using the equation

ED=2*{square root over ((GA lesion area/π)}

In a fourth aspect, a method for treating a patient with geographic atrophy lesions is provided. In embodiments, the method comprises identifying a lesion associated with geographic atrophy in a patient, the lesion having a lesion size; and administering or instructing to administer a drug or therapeutic agent that slows progression of geographic atrophy lesion growth in a therapeutically effective dose to slow the rate of lesion growth in a population of subjects with a baseline geographic atrophy lesion area of greater than or equal to 8 mm² relative to placebo- or sham-treated subjects in a population of subjects with a baseline geographic atrophy lesion area of greater than or equal to 8 mm². In embodiments, the drug or therapeutic agent is administered in a therapeutically effective dose to slow the rate of lesion growth in a population of subjects with a baseline lesion effective diameter of greater than or equal to 3.19 mm relative to placebo- or sham-treated subjects in a population of subjects with a baseline lesion effective diameter of greater than or equal to 3.19 mm. In embodiments, the drug and composition are as described above.

In addition to the exemplary aspects and embodiments described above, further aspects and embodiments will become apparent by reference to the drawings and by study of the following descriptions.

Additional embodiments of the present methods, treatments and compositions, and the like, will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present invention. Additional aspects and advantages of the present invention are set forth in the following description and claims, particularly when considered in conjunction with the accompanying examples and drawings.

### BRIEF DESCRIPTION OF THE DRAWINGS

FIG. 1 is a graph showing the geographic atrophy area progression rate (mm²/year) for subjects having a baseline geographic atrophy lesion area (mm²) for a sham treatment (○), a brimonidine tartrate implant formulation containing the equivalent of 132 μg brimonidine free base as described in EXAMPLE 1 (□) and a brimonidine tartrate implant formulation containing the equivalent of 264 μg brimonidine free base as described in EXAMPLE 1 (Δ).

FIG. 2 is a graph showing the change in GA lesion effective diameter (mm) over time (months) for a sham treatment (○), a brimonidine tartrate implant formulation containing the equivalent of 132 μg brimonidine free base as described in EXAMPLE 1 (□) and a brimonidine tartrate implant formulation containing the equivalent of 264 μg brimonidine free base as described in EXAMPLE 1 (Δ) for subjects having a baseline GA lesion area of greater than or equal to 9 mm².

FIG. 3 is a graph showing a comparison of a population of patients showing the change in GA lesion effective diameter (mm) over time (months) for a population having a baseline GA lesion area of <9 mm² (○) and a population having a GA lesion area of greater than or equal to 9 mm² (Δ).

FIGS. 4A-4B are graphs showing the association between the baseline geographic lesion area, geographic lesion perimeter, geographic atrophy circularity index, and rate of geographic lesion progression at month 12 (FIG. 4A) and at month 24 (FIG. 4B).

### DETAILED DESCRIPTION

### I. Definitions

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7μm are also explicitly disclosed, as well as the range of values greater than or equal to 1 μm and the range of values less than or equal to 8 μm.

The singular forms “a,” “an,” and “the” include plural referents unless the context clearly dictates otherwise. Thus, for example, reference to a “polymer” includes a single polymer as well as two or more of the same or different polymers, reference to an “excipient” includes a single excipient as well as two or more of the same or different excipients, and the like.

The word “about” when immediately preceding a numerical value means a range of plus or minus 10% of that value, e.g., “about 50” means 45 to 55, “about 25,000” means 22,500 to 27,500, etc., unless the context of the disclosure indicates otherwise, or is inconsistent with such an interpretation. For example, in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

The compositions of the present disclosure can comprise, consist essentially of, or consist of, the components disclosed.

All percentages, parts and ratios are based upon the total weight of the topical compositions and all measurements made are at about 25° C., unless otherwise specified.

The terms “biodegradable polymer” or “bioerodible polymer” refer to a polymer or polymers which degrade or erode in vivo, and wherein degradation or erosion of the polymer or polymers over time occurs concurrent with and/or subsequent to the release of a therapeutic agent. A biodegradable polymer may be a homopolymer, a copolymer, or a polymer comprising more than two polymeric units. In some embodiments, a “biodegradable polymer” may include a mixture of two or more homopolymers or copolymers.

The phrase “pharmaceutically acceptable” is employed herein to refer to those compounds, salts, compositions, dosage forms, etc., which are—within the scope of sound medical judgment—suitable for use in contact with the tissues of human beings and/or other mammals without excessive toxicity, irritation, allergic response, or other problem or complication, commensurate with a reasonable benefit/risk ratio. In some, but not all, aspects, “pharmaceutically acceptable” means approved by a regulatory agency of the federal or a state government, or listed in the U.S. Pharmacopeia or other generally recognized pharmacopeia for use in mammals (e.g., animals), and more particularly, in humans.

The terms “treat”, “treating”, or “treatment” as used herein, refer to reduction or resolution or prevention of an ocular condition, ocular injury or damage, or to promote healing of injured or damaged ocular tissue.

The term “therapeutically effective amount” as used herein, refers to the level or amount of a therapeutic agent needed to treat an ocular condition, reduce or prevent the symptoms of an ocular condition, or reduce or prevent ocular injury or damage.

The terms “inhibiting” or “reducing” are used in reference to methods to inhibit or to reduce lesion size (area or effective diameter) in a population as compared to a placebo-or sham-treated population.

As used herein, an “intraocular implant” refers to a device or elements that is structured, sized, or otherwise configured to be placed in an eye. Intraocular implants are generally biocompatible with physiological conditions of an eye. Intraocular implants may be placed in an eye without disrupting vision of the eye.

As used herein, an “ocular condition” is a disease ailment or condition which affects or involves the eye or one of the parts or regions of the eye. The eye can include the eyeball and the tissues and fluids that constitute the eyeball, the periocular muscles (such as the oblique and rectus muscles) and the portion of the optic nerve which is within or adjacent the eyeball.

An “anterior ocular condition” is a disease, ailment, or condition which affects or which involves an anterior (i.e. front of the eye) ocular region or site, such as a periocular muscle, an eye lid or an eye ball tissue or fluid which is located anterior to the posterior wall of the lens capsule or ciliary muscles. Thus, an anterior ocular condition can affect or involve the conjunctiva, the cornea, the anterior chamber, the iris, the posterior chamber (located behind the retina, but in front of the posterior wall of the lens capsule), the lens or the lens capsule and blood vessels and nerve which vascularize or innervate an anterior ocular region or site.

A “posterior ocular condition” is a disease, ailment or condition which primarily affects or involves a posterior ocular region or site such as choroid or sclera (in a position posterior to a plane through the posterior wall of the lens capsule), vitreous, vitreous chamber, retina, optic nerve or optic disc, and blood vessels and nerves that vascularize or innervate a posterior ocular region or site.

By reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by reserving the right to proviso out or exclude any individual substituents, analogs, compounds, ligands, structures, or groups thereof, or any members of a claimed group, less than the full measure of this disclosure can be claimed for any reason.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

### II. Methods of Treatment

The prevalence of geographic atrophy (GA) in the US is estimated at 650,000 individuals over the age of 80, representing 6.9%. Currently, there are no approved treatments for GA. In one aspect, a method of treating AMD is described herein. In embodiments, the methods are useful for treating dry AMD. In embodiments, the methods are particularly useful for treating advanced stages of dry AMD such as GA.

GA refers to the clinical condition of a nearly complete loss or atrophy of a discrete area of the RPE cells under the retina. GA often develops initially in the region near the fovea. The areas of atrophy may develop as several small areas (lesions) that tend to enlarge and possibly coalesce over time. The GA lesions grow slowly over time (about 1.3-2.6 mm² per year) and may result in loss of central vision.

A problem with methods of treating GA is the slow progression (rate of growth) of lesion size which hampers the ability to determine efficacy and/or establish appropriate dosing. Because effects on the rate of progression are seen over years rather than months or even one year, the effect of a treatment is difficult to determine and adjust as needed. In order to address these problems, the treatments described herein are used for patients showing a rate of progression for lesion size or area such that differences achieved with administration of a therapeutic agent may be determined and/or adjusted on a clinically meaningful timeline. In embodiments, the patient population is selected such that an effect is seen at about twelve months or less. This effect is of great benefit as previous studies may require 18-24 months to see any effect due to the slow growth rate or progression of the lesion.

In one embodiment, a treatment method for slowing or halting the rate of growth of a lesion associated with geographic atrophy is provided. In some embodiments, a therapeutically effective amount of one or more therapeutic agents is administered to a subject having a baseline geographic atrophy lesion area of greater than or equal to about 8 mm². In embodiments, administering the therapeutic agent is effective to slow or halt the rate of lesion growth in a population of subjects with a baseline geographic atrophy lesion area of greater than or equal to 8 mm² relative to placebo-treated subjects in a population of subjects with a baseline geographic atrophy lesion area of greater than or equal to 8 mm². In some embodiments, the therapeutic agent is administered to a subject having a baseline geographic atrophy lesion area of at least or greater than about 8-50 mm². In some embodiments, the therapeutic agent is administered to a subject having a baseline geographic atrophy lesion area of at least or greater than about 8-9 mm², 8-10 mm², 8-11 mm², 8-12 mm², 8-13 mm², 8-14 mm², 8-15 mm², 8-16 mm², 8-17 mm², 8-18 mm², 8-19 mm², 8-20 mm², 8-25 mm², 8-30 mm², 8-40 mm², 9-10 mm², 9-11 mm², 9-12 mm², 9-13 mm², 9-14 mm², 9-15 mm², 9-16 mm², 9-17 mm², 9-18 mm², 9-19 mm², 9-20 mm², 9-25 mm², 9-30 mm², 9-40 mm², 9-50 mm², 10-11 mm², 10-12 mm², 10-13 mm², 10-14 mm², 10-15 mm², 10-16 mm², 10-17 mm², 10-18 mm², 10-19 mm², 10-20 mm², 10-25 mm², 10- 30 mm², 10-40 mm², 10-50 mm², 11-12 mm², 11-13 mm², 11-14 mm², 11-15 mm², 11-16 mm², 11-17 mm², 11-18 mm², 11-19 mm², 11-20 mm², 11-25 mm², 11-30 mm², 11-40 mm², 11-50 mm², 12-13 mm², 12-14 mm², 12-15 mm², 12-16 mm², 12-17 mm², 12-18 mm², 12-19 mm², 12-20 mm², 12-25 mm², 12-30 mm², 12-40 mm², 12-50 mm², 13-14 mm², 13-15 mm², 13-16 mm², 13-17 mm², 13-18 mm², 13-19 mm², 13-20 mm², 13-25 mm², 13-30 mm², 13-40 mm², 13-50 mm², 14-15 mm², 14-16 mm², 14-17 mm², 14-18 mm², 14-19 mm², 14-20 mm², 14-25 mm², 14-30 mm², 14-40 mm², 14-50 mm², 15-16 mm², 15-17 mm², 15-18 mm², 15-19 mm², 15-20 mm², 15-25 mm², 15-30 mm², 15-40 mm², 15-50 mm², 16-17 mm², 16-18 mm², 16-19 mm², 16-20 mm², 16-25 mm², 16-30 mm², 16-40 mm², 16-50 mm², 17-18 mm², 17-19 mm², 17-20 mm², 17-25 mm², 17-30 mm², 17-40 mm², 17-50 mm², 18-19 mm², 18-20 mm², 18-25 mm², 18-30 mm², 18-40 mm², 18-50 mm², 19-20 mm², 19-25 mm², 19-30 mm², 19-40 mm², 19-50 mm², 20-25 mm², 20-30 mm², 20-40 mm², 20-50 mm², 25-30 mm², 25-40 mm², 25-50 mm², 30-40 mm², 30-50 mm², or 40-50 mm². In some embodiments, the therapeutic agent is administered to a subject having a baseline geographic atrophy lesion area of at least or greater than about 9 mm², about 10 mm², about 11 mm², about 12 mm², about 13 mm², about 14 mm², about 15 mm², about 20 mm², about 25 mm², about 30 mm², about 40 mm², about 50 mm² or more.

It will be appreciated that the geographic atrophy lesion area may be measured by any suitable method as known in the art. In some non-limiting embodiments, the lesion area is measured by one or more of color fundus photography and/or fundus autofluorescence.

In another embodiment, a method of slowing the progression of lesion size associated with geographic atrophy is provided. In some embodiments, a drug or therapeutic agent that slows progression of lesion size associated with geographic atrophy is administered to a subject. In embodiments, a therapeutically effective dose of the drug is established by dosing a population of subjects with a baseline geographic atrophy lesion area of greater than or equal to about 8 mm². In some embodiments, the therapeutic agent is administered to a subject having a baseline geographic atrophy lesion area of greater than about 9 mm², about 10 mm², about 11 mm², about 12 mm², about 13 mm², about 14 mm², about 15 mm², or more.

In another embodiment, a method for treating a patient with geographic atrophy lesions is provided. In embodiments, the method comprises identifying a lesion associated with geographic atrophy in a patient, the lesion having a lesion size and administering or instructing a subject/provider to administer a drug that slows progression of geographic atrophy lesion growth in a therapeutically effective dose to slow rate of lesion growth in a population of subjects with a baseline geographic atrophy lesion area of greater than or equal to 8 mm² relative to placebo-treated subjects in a population of subjects with a baseline geographic atrophy lesion area of greater than or equal to 8 mm². In some embodiments, the therapeutic agent is administered to a subject having a baseline geographic atrophy lesion area of at least or greater than about 8-50 mm². In some embodiments, the therapeutic agent is administered to a subject having a baseline geographic atrophy lesion area of at least or greater than about 8-9 mm², 8-10 mm², 8-11 mm², 8-12 mm², 8-13 mm², 8-14 mm², 8-15 mm², 8-16 mm², 8-17 mm², 8-18 mm², 8-19 mm², 8-20 mm², 8-25 mm², 8-30 mm², 8-40 mm², 9-10 mm², 9-11 mm², 9-12 mm², 9-13 mm², 9-14 mm², 9-15 mm², 9-16 mm², 9-17 mm², 9-18 mm², 9-19 mm², 9-20 mm², 9-25 mm², 9-30 mm², 9-40 mm², 9-50 mm², 10-11 mm², 10-12 mm², 10-13 mm², 10-14 mm², 10-15 mm², 10-16 mm², 10-17 mm², 10-18 mm², 10-19 mm², 10-20 mm², 10-25 mm², 10- 30 mm², 10-40 mm², 10-50 mm², 11-12 mm², 11-13 mm², 11-14 mm², 11-15 mm², 11-16 mm², 11-17 mm², 11-18 mm², 11-19 mm², 11-20 mm², 11-25 mm², 11-30 mm², 11-40 mm², 11-50 mm², 12-13 mm², 12-14 mm², 12-15 mm², 12-16 mm², 12-17 mm², 12-18 mm², 12-19 mm², 12-20 mm², 12-25 mm², 12-30 mm², 12-40 mm², 12-50 mm², 13-14 mm², 13-15 mm², 13-16 mm², 13-17 mm², 13-18 mm², 13-19 mm², 13-20 mm², 13-25 mm², 13-30 mm², 13-40 mm², 13-50 mm², 14-15 mm², 14-16 mm², 14-17 mm², 14-18 mm², 14-19 mm², 14-20 mm², 14-25 mm², 14-30 mm², 14-40 mm², 14-50 mm², 15-16 mm², 15-17 mm², 15-18 mm², 15-19 mm², 15-20 mm², 15-25 mm², 15-30 mm², 15-40 mm², 15-50 mm², 16-17 mm², 16-18 mm², 16-19 mm², 16-20 mm², 16-25 mm², 16-30 mm², 16-40 mm², 16-50 mm², 17-18 mm², 17-19 mm², 17-20 mm², 17-25 mm², 17-30 mm², 17-40 mm², 17-50 mm², 18-19 mm², 18-20 mm², 18-25 mm², 18-30 mm², 18-40 mm², 18-50 mm², 19-20 mm², 19-25 mm², 19-30 mm², 19-40 mm², 19-50 mm², 20-25 mm², 20-30 mm², 20-40 mm², 20-50 mm², 25-30 mm², 25-40 mm², 25-50 mm², 30-40 mm², 30-50 mm², or 40-50 mm². In some embodiments, the therapeutic agent is administered to a subject having a baseline geographic atrophy lesion area of at least or greater than about 9 mm², about 10 mm², about 11 mm², about 12 mm², about 13 mm², about 14 mm², about 15 mm², about 20 mm², about 25 mm², about 30 mm², about 40 mm², about 50 mm² or more.

In embodiments, the therapeutic agent is selected from lampalizumab, flucinolone acetonide, ORACEA®, emixustat hydrochloride, sirolimus, MC-1101, Zimura®, and brimonidine or a salt thereof. In one embodiment, the therapeutic agent comprises brimonidine or a salt thereof. Brimonidine (5-bromo-6-(2-imidazolidinylideneamino) quinoxaline) is an alpha-2-selective adrenergic receptor agonist that has been found to be effective for treating open-angle glaucoma by decreasing aqueous humor production and increasing uveoscleral outflow. Brimonidine tartrate ophthalmic solution 0.2% (marketed as ALPHAGAN®) was approved by the US Food and Drug Administration (FDA) in September 1996 and in Europe in March 1997 (United Kingdom).

A neuroprotective effect of brimonidine tartrate has been shown in animal models of optic nerve crush, moderate ocular hypertension, pressure-induced ischemia, and vascular ischemia. The neuroprotective effect of topical applications of brimonidine tartrate has also been explored clinically in patients with glaucoma, age-related macular degeneration, retinitis pigmentosa, diabetic retinopathy, and acute non-arteritic anterior ischemic optic neuropathy.

Brimonidine is also publicly available as brimonidine free base. Brimonidine free base is generally hydrophobic. In some embodiments, the therapeutic agent is brimonidine free base. In some embodiments, the therapeutic agent is a pharmaceutically acceptable acid addition salt of brimonidine. One exemplary salt is brimonidine tartrate (AGN 190342-F, 5-bromo-6-(2-imidazolidinylideneamino) quinoxaline tartrate). Both brimonidine free base and brimonidine tartrate are chemically stable and have melting points higher than 200° C.

The therapeutic agent may be administered by any suitable method. In some embodiments, the therapeutic agent is administered to a posterior segment of the eye. In embodiments, the methods comprise administering the therapeutic agent by injection such as, for example, at least one of intravitreal injection, subconjuctival injection, subtenon injection, retrobulbar injection, and suprachoroidal injection.

In some embodiments, the therapeutic agent is administered in a sustained release implant such as described in U.S. Pat. Nos. 8,969,415 and 9,610,246, both of which are incorporated herein by reference. In embodiments, the implant is a solid intraocular implant comprising an active agent such as brimonidine or a salt thereof and a biodegradable polymer matrix.

According to some embodiments, implants can be formulated with particles of the brimonidine or a salt thereof dispersed within the bioerodible polymer matrix. According to some embodiments, the implants can be monolithic, having the therapeutic agent homogenously distributed through the biodegradable polymer matrix, or encapsulated, where a reservoir of active agent is encapsulated by the polymeric matrix. In some embodiments, the therapeutic agent may be distributed in a non-homogeneous pattern in the biodegradable polymer matrix. For example, in an embodiment, an implant may include a first portion that has a greater concentration of the therapeutic agent (such as brimonidine or a salt thereof) relative to a second portion of the implant.

Examples of suitable polymeric materials for the polymer matrix include, without limitation, polyesters. For example, polymers of D-lactic acid, L-lactic acid, poly(D,L-lactide), racemic lactic acid, glycolic acid, polycaprolactone, and combinations thereof may be used for the polymer matrix. In one embodiment, the polymer is a poly (D,L-lactide-co-glycolide) polymer. In some embodiments, a polyester, if used, may be a homopolymer, a copolymer, or a mixture thereof. In some embodiments, the implant comprises one or more copolymers of glycolic acid and lactic acid where the rate of biodegradation can be controlled, in part, by the ratio of glycolic acid to lactic acid. The mol percentage (% mol) of polylactic acid in the polylactic acid polyglycolic acid (PLGA) copolymer can be between 15 mol % and about 85 mol %. In some embodiments, the mol percentage of polylactic acid in the (PLGA) copolymer is between about 35 mol % and about 65 mol %. In some embodiments, a PLGA copolymer with 50 mol % polylactic acid and 50 mol % polyglycolic acid can be used in the polymer matrix. In embodiments, the biodegradable polymer matrix of the intraocular implant comprises a mixture of two or more biodegradable polymers.

In some embodiments, the implant is comprised of between about 30-80 wt % of the biodegradable polymer. In other embodiments, the implant is comprised of between about 30-40 wt %, 30-50 wt %, 30-60 wt %, 30-70 wt %, 30-75 wt %, 40-50 wt %, 40-60 wt %, 40-70 wt %, 40-75 wt %, 40-80 wt %, 50-60 wt %, 50-70 wt %, 50-75 wt %, 50-80 wt %, 60-70 wt %, 60-75 wt %, 60-80 wt %, 70-75 wt %, or 75-80 wt %. In one embodiment, the implant is comprised of about 50-65 wt % of the biodegradable polymer(s). In embodiments, the implant is comprised of at least about 40 wt %, 50 wt %, 60 wt %, 65 wt %, 70 wt %, 75 wt %, or 80 wt % of the one or more biodegradable polymers. In other embodiments, the implant is comprised of up to about 40 wt %, 50 wt %, 60 wt %, 65 wt %, 70 wt %, 75 wt %, or 80 wt % of the one or more biodegradable polymers.

In some embodiments, the implant is comprised of between about 20-70 wt % of the one or more therapeutic agents. In other embodiments, the implant is comprised of between about 20-25 wt %, 20-30 wt %, 20-40 wt %, 20-50 wt %, 20-60 wt %, 25-30 wt %, 25-40 wt %, 25-50 wt %, 25-60 wt %, 25-70 wt %, 30-40 wt %, 30-50 wt %, 30-60 wt %, 30-70 wt %, 40-50 wt %, 40-60 wt %, 40-70 wt %, 50-60 wt %, 50-70 wt %, or 60-70 wt %. In embodiments, the implant is comprised of at least about 20 wt %, 25 wt %, 30 wt %, 40 wt %, 50 wt %, 60 wt %, or 70 wt % of the one or more therapeutic agents. In other embodiments, the implant is comprised of up to about 20 wt %, 25 wt %, 30 wt %, 40 wt %, 50 wt %, 60 wt %, or 70 wt % of the one or more therapeutic agents.

In some embodiments herein, the implant can provide for extended release time of one or more therapeutic agent or agents. Thus, for example, a patient who has received such an implant in their eye can receive a therapeutic amount of an agent for a long or extended time period without requiring additional administrations of the agent. According to some embodiments an implant may also only remain within the eye of a patient for a targeted or limited amount of time before it degrades completely or nearly completely. By limiting the amount of time a foreign object, such as an implant is in a patient's eye or vitreous, a patient's comfort is optimized and their risk for infection or other complications is minimized. Also, complications that may arise from an implant colliding with the cornea or other part of the eye in the dynamic fluid of the vitreous can be avoided.

### III. Methods of Assessing Treatments for Geographic Atrophy

The progression of GA has previously been measured clinically by visual function tests. However, these tests rely on subjective measurements and are difficult to standardize for clinical end points. Further, visual acuity and/or function outcomes may be insensitive to a slow progression of the GA. Alternative end points have been considered as end points for clinical trials including decreasing the rate of progression of GA lesion enlargement. The US Food and Drug Administration has adopted the anatomic end point of GA enlargement rate as a main outcome parameter in clinical trials. In these methods, improvement over a placebo or sham treatment may be shown by slowing or stopping the lesion growth. The selection of suitable outcome and end points (e.g. primary and/or secondary outcomes) is a critical factor in the design of a clinical study. However, due to the slow growth or progression of GA lesion size, current methods require multiple years of evaluation (e.g. 18-24 months) in order to determine if an intervention slows the rate of progression. The present method uses patient selection criteria (e.g. lesion area) in order to allow effective determination or estimation of drug effect at or about 12 months or less.

Studies have shown wide variability in the growth rates of GA lesions between individuals (Sunness et al., Retina, 27 (2):204-210, 2007). In a large pool of patients with GA, sufficient lesion expansion for the placebo/sham comparator may take several years in order to show sufficient difference between treatment and sham effects in order to evaluate a treatment. Baseline GA lesion area has been shown to correlate with GA progression rate (Schmitz-Valckenberg, et al., Ophthalmology, 123:361-368, 2016), with patients having larger lesions progressing more rapidly. Selecting subjects with a suitable growth rate (e.g. rapidly or more rapidly progressing disease) allows for faster evaluation of the treatment under investigation. Treatment with a placebo or sham treatment is expected to have approximately linear growth of the GA lesion area over limited periods of time. Treatment with an effective therapeutic agent is expected to show the divergence of the GA lesion area for treatments. In order to show effectiveness of a treatment, a significant difference between GA lesion area or effective diameter for the sham treatment versus the drug treatment may need to be clinically meaningful. In embodiments, a difference of about 5-25% is found between the GA lesion area or effective diameter for the sham treatment as compared to the drug treatment. In some embodiments, a difference of about 5-10%, 5-15%, 5-20%, 10-15%, 10-20%, 10-25%, 15-20%, 15-25%, and 20-25% difference between the GA lesion area or effective diameter for the sham treatment as compared to the drug treatment. In embodiments, a difference of at least about 5%, 10%, 15%, 20% or 25% is found between the GA lesion area or effective diameter for the sham treatment as compared to the drug treatment is found. In embodiments, a difference of up to about 5%, 10%, 15%, 20% or 25% is found between the GA lesion area or effective diameter for the sham treatment as compared to the drug treatment is found.

Selecting subjects for investigation having a baseline GA lesion area of at least about 8 mm² provides a suitably rapid progression of placebo- or sham-treated subjects to allow evaluation of a therapeutic on a reduced time scale. The rapid progression of the placebo- or sham-treated subjects permits earlier detection of a window between placebo/sham and treatment groups. Selection of patients having a baseline GA lesion area of at least about 8 or 9 mm² allows for evaluation of the therapeutic effect in a reduced time period, e.g. about 12 months or less. The value of selecting or including patients for a study having the disclosed GA lesion area is that these patients have been found to show a high enough rate of progression for the lesion area that allows effective estimation of the therapeutic effects of an investigational agent in about 12 months or less. In embodiments, the investigational method permits a detection window of about 6 months to less than two years, which is a significant reduction than conventional studies of GA, which generally are designed with an 18 month to 2-year primary endpoint. In some embodiments, the investigational method permits a detection window or evaluation endpoint of about 6-12 months, 6-11 months, 6-10 months, 6-9 months, 6-8 months, 6-7 months, 7-12 months, 7-11 months, 7-10 months, 7-9 months, 7-8 months, 8-12 months, 8-11 months, 8-10 months, 8-9 months, 9-12 months, 9-11 months, 9-10 months, 10-12 months, 10-11 months, or 11-12 months. In some embodiments, the investigational method permits a detection window or investigational endpoint of up to about 6, 7, 8, 9, 10, 11, or 12 months or less. In some embodiments, the investigational methods described herein permit a detection window or investigational endpoint of less than about 18 or 24 months.

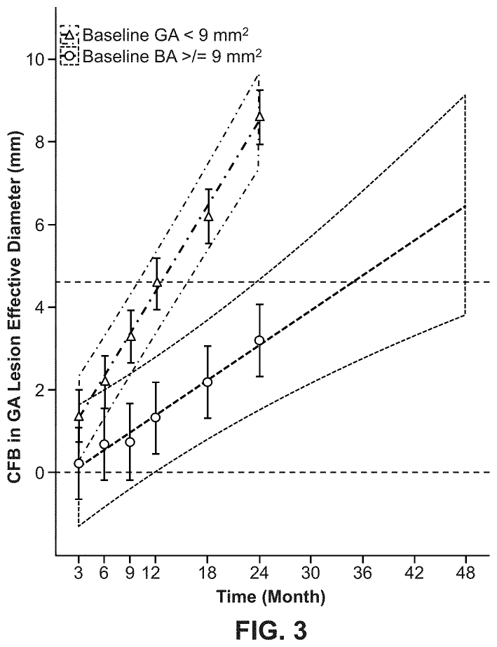

The methods for evaluating a treatment protocol or method for GA involves the selection patients having a baseline GA lesion area of greater or equal to about 8 mm² or about 9 mm². FIG. 3 is a graph showing the GA lesion effective diameter (in mm) over 24 months for subjects administered a sham treatment as described in Example 2. The change from baseline in GA lesion effective diameter over twelve months of treatment and for patients having a baseline GA lesion area of less than 9 mm² (Δ) or greater than or equal to 9 mm² (○) is shown. The GA lesion area for the subjects was observed without treatment for an additional 12 months. The patients having a baseline GA lesion area of at least 9 mm² showed a significantly higher progression of lesion effective diameter as compared to the patients having a baseline of less than 9 mm², even in one year. The sham treated subjects that also have a larger GA lesion area at the outset have a rapid progression that permits earlier detection and comparison of the effect of treatment on the GA lesion area. The patients having a baseline GA lesion area of at least 9 mm² showed a window between the sham and treatments at least within the year of treatment. This window is at least 50% faster than conventional study designs that use a two-year primary timepoint due to the slow rate of progression for the treatment and/or sham administration.

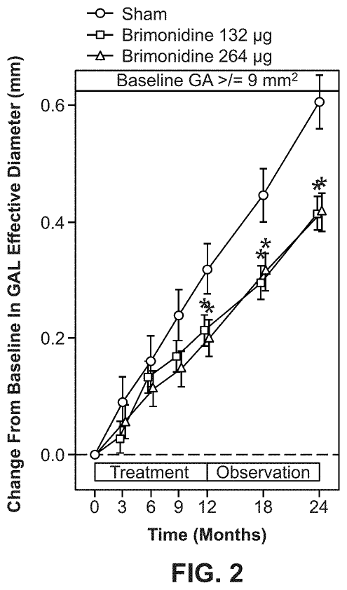

The subjects having a baseline GA lesion area of >9 mm² progressed 4.6 mm² during the 12 months of treatment. In comparison, subjects with a low baseline GA area of >9 mm² would take approximately 36 months to progress by the same amount from baseline. In this example, selecting a population of subjects having a suitable GA lesion size allows for assessment of an investigational treatment two years earlier (e.g. 12 months rather than 36 months). Thus, subjects having a medium/low baseline GA lesion area would show any difference due to treatment significantly faster (e.g. at least about 30-50%) than subjects having a low baseline GA lesion area. As seen in FIG. 2, selecting the subjects having a baseline GA lesion area of greater than or equal to 9mm² allows for the effect of treatment with each of a brimonidine tartrate implant with a formulation as described in EXAMPLE 1 having the equivalent of brimonidine free base at 132 μg (□) or the equivalent of 264 μg brimonidine free base (Δ) to be detectable as compared to the sham treated subjects (○) even within the 12-month treatment period. By selecting subjects having a baseline lesion area of sufficient size, the lesions grow at a rate that is suitable for comparison to a placebo or sham comparator.

In embodiments, data from the investigational study is transformed using a square root transformation and expressed as the clinically meaningful term, effective diameter using the process as described in Feuer et al. (JAMA Ophthalmol, 131 (1):110-111, 2013) (see also Kim et al., ARVO 2017 abstract). In embodiments, a square root transformation of baseline GA area appears to ameliorate the effect of baseline area on GA progression, which could simplify clinical trial enrollment and analysis. Using the square root of the lesion area improves the statistical properties of GA lesion area data. In embodiments, square root transformation of the lesion area data improves distribution of the data (more normal) and/or the association between the baseline GA area and the rate of progression is less apparent. (see Feuer) In embodiments, the GA lesion area is transformed to an effective diameter (ED) using the equation:

ED=2*{square root over ((GA lesion area/π)}

Thus, in embodiments, the subjects are selected such that the baseline effective diameter of the GA lesion is at least or greater than about 3.19-7.98 mm, or at least or greater than about 3.38-7.98 mm.

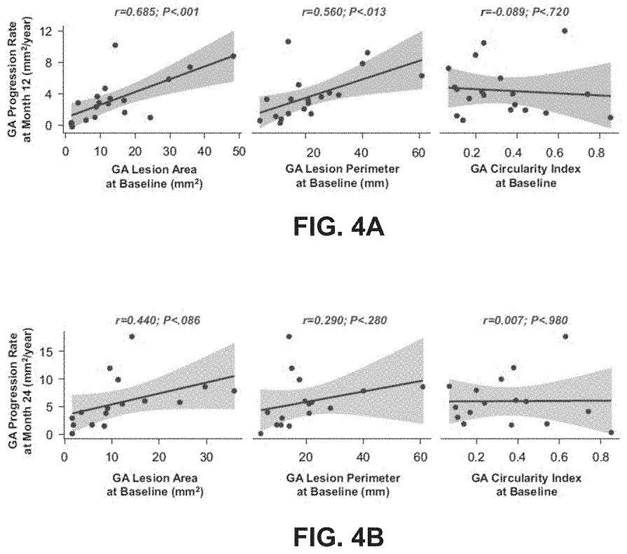

In some embodiments, the baseline lesion perimeter is used as an alternative predictive measure of progression rate. Perimeter represents an indication of the amount of cells at risk for GA progression, and, therefore provides predictive value for the progression rate. The lesion perimeter may be measured by any suitable method as known in the art including, but not limited to one or more of color fundus photography and/or fundus autofluorescence. FIGS. 4A-4B shows a graph of the GA progression rate (mm²/year) at 12 or 24 months for subjects having a GA lesion perimeter of 0-60 mm. As seen in the figures, selection of subjects having a sufficiently high growth rate allows for effective estimation of the effect of a therapeutic agent after one month.

The GA Circularity Index (GACI), an indicator of lesion shape irregularity calculated as the ratio of lesion area and the area of the circle defined by the lesion perimeter. In some embodiments, the GACI is available as to model, monitor or predict lesion progression rate. A higher GACI value predicts slower rate of progression. The GACI is defined as the ratio of the Measured GA Area to the Expected Area (EA) and has a range of 0.0 to 1.0. EA is defined as the squared perimeter (P) divided by 4π, which is derived from the geometric formula for the perimeter (2π) and area of a circle (πr²): GACI=Measured GA Area/EA where EA=P²/4π.

It will be appreciated that the therapeutic agent for the investigational study is not limited and may be any drug, compound or other agent for investigating the clinical effectiveness in treating, delaying or preventing the progression of geographic atrophy.

### IV. Examples

The following examples are illustrative in nature and are in no way intended to be limiting.

### EXAMPLE 1

### Administration of Intraocular Implant Comprising Brimonidine Tartrate

Intraocular implants comprising 400 μg or 200 μg brimonidine tartrate were prepared by blending the brimonidine tartrate with one or more biodegradable polymers. The resulting powder was extruded into filaments that were cut to form implants with the target weight of 400 μg or 200 μg brimonidine tartrate to provide an equivalent brimonidine free base dose of 132 μg or 264 μg, respectively. Table 1 provides the implant formulations.

TABLE 1
Brimonidine tartrate implant formulations

| | | Formulation (% w/w) | | | | | |
|---|---|---|---|---|---|---|---|
| Brimonidine | | | | Poly D,L- | Approximate Implant | | |
| Tartrate | BFB | | Poly | lactide | Physical Characteristics | | |
| Dose | Dose | Brimonidine | D,L- | (high | Diameter | Length | Weight |
| (μg) | (μg) | Tartrate | lactide | MW) | (μm) | (μm) | (μm) |
| 200 | 132 | 35 | 40 | 25 | 460 | 2.8 | 571 |
| 400 | 264 | 35 | 40 | 25 | 460 | 5.6 | 1143 |

BFB is the dose of brimonidine free base provided by the implant.

The implants are sterilized by loading into 25 G applicators and gamma-sterilized at 25 to 40 kGy dose. The potency per implant may be confirmed by a HPLC assay.

An implant comprising brimonidine tartrate or a sham treatment (no drug) was intravitreally inserted into the posterior segment of the eye using a 22-gauge insert.

The geographic atrophy lesion area and/or lesion perimeter is measured by a suitable method such as color fundus photography about 12 months after administering the implant.

### EXAMPLE 2

### Administration of Intraocular Implant Comprising Brimonidine

Biodegradable implants comprising 200 μg or 400 μg brimonidine tartrate, to provide an equivalent dose of 132 μg or 264 μg brimonidine free base, in a 22-gauge implant were prepared according to the method as described in Example 1. The polymer matrix formulation comprises 50% w/w brimonidine free base, 25% w/w poly (D,L-lactide), acid end (intrinsic viscosity 0.16-0.24 dL/g; low molecular weight), 25% w/w 75:25 poly(D,L-lactide-co-glycolide (intrinsic viscosity 0.16-0.24 dL/g; low molecular weight). The implant has a diameter of 356 length ˜6 and weight 800 μg.

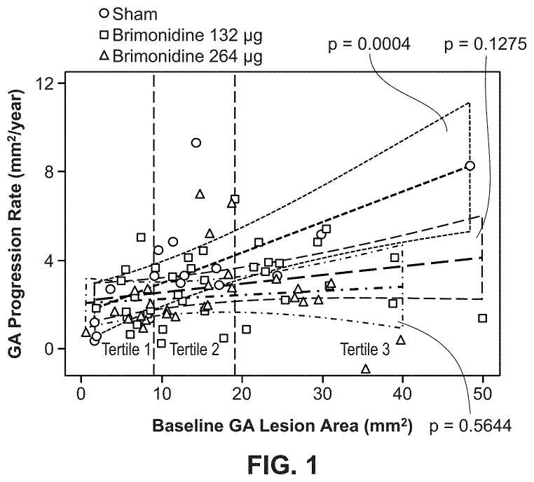

The baseline geographic atrophy area in at least one affected eye was determined for each subject diagnosed with geographic atrophy secondary to age-related macular degeneration. An brimonidine tartrate-containing implant comprising the equivalent of 132 μg brimonidine free base dose (49 subjects), brimonidine tartrate-containing implant the equivalent of 264 μg brimonidine free base dose (41 subjects), or a sham (no drug) (23 subjects) was intravitreally inserted into the posterior segment of the eye of a subject having geographic atrophy in the treatment eye. The treatment was repeated every 3 months for 12 months. The geographic atrophy lesion area was measured by color fundus photography. FIG. 1 shows the progression rate (mm²/year) for the sham and drug treatments as compared to the baseline GA lesion (mm²) for subjects administered a brimonidine tartrate-containing implant the equivalent of 132 μg brimonidine free base (□) or a brimonidine tartrate-containing implant the equivalent of 264 μg brimonidine free base (Δ).

The treatment subjects having a lower GA area baseline (tertile T1->0.5-9 mm²) had a lower GA progression rate for the year for both the treatment doses. The treatment subjects having a medium GA area baseline (tertile T2->9-19 mm²) had a slightly higher GA progression rate for the year with the low dose having a higher progression rate than the high dose treatment. The treatment subjects having a high GA area baseline (tertile T3->19-50 mm²) had an even higher GA progression rate for the year with the low dose having a faster progression rate than the high dose treatment. Some of the subjects had negative change from baseline values representing stable lesions at the one-year timepoint.

Using R software (R Studio), matrix correlations were conducted to assess GA progression rate at month 12 and month 24 as a function of baseline GA area, GACI, and perimeter with the results shown in FIGS. 4A-4B.

The baseline GA lesion area (mm²), GA Lesion Perimeter (mm), and GACI means (range) were 14.38 (1.65-48.34) mm², 21.88 (4.15-60.72) mm, and 0.33 (0.07-0.85), respectively, for the sham treatment group. In these patients, GA progression rates (mean change from baseline±standard error of the mean) were 3.31±0.67 mm²/year at month 12 and 5.90±1.13 mm²/year at month 24. Baseline GA lesion area and GA Lesion Perimeter demonstrated statistically significant associations with GA progression rate at month 12 (r=0.685; P<0.001 and r=0.560; P<0.013, respectively). Associations at month 24 followed the same trends (FIG. 4B).

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

It is claimed:

1. A treatment method for slowing rate of growth of a lesion associated with geographic atrophy, comprising:
administering to a patient a composition comprising a therapeutically effective amount of a drug to slow rate of growth of a lesion associated with geographic atrophy, wherein the therapeutically effective amount slows rate of lesion growth in a population of subjects with a baseline geographic atrophy lesion area of greater than or equal to 8 mm² relative to placebo-treated subjects in a population of subjects with a baseline geographic atrophy lesion area of greater than or equal to 8 mm².

2. The method of claim 1, wherein the drug is brimonidine or a salt thereof.

3. The method of claim 2, wherein brimonidine is selected from brimonidine free base or brimonidine tartrate.

4. The method of claim 3, wherein the composition is an ocular implant.

5. The method of claim 4, wherein the ocular implant is a solid ocular implant.

6. The method of claim 5, wherein the solid ocular implant is comprised of a biodegradable polymer.

7. The method of claim 6, wherein the biodegradable polymer is poly (D,L-lactide).

8. The method of claim 7, wherein the solid intraocular implant is comprised of between about 50-65 wt % of biodegradable polymer.

9. The method of claim 8, wherein the ocular implant comprises a poly (D,L-lactide-co-glycolide) polymer.

10. The method of claim 9, wherein the composition comprises between about 25-60 wt % drug.

11. A method of slowing progression of lesion size associated with geographic atrophy, comprising:
administering to a patient a drug that slows progression of lesion size associated with geographic atrophy, wherein a therapeutically effective dose of the drug was established by dosing a population of subjects with a baseline geographic atrophy lesion area of greater than or equal to about 8 mm².

12. A method to evaluate a drug for use in reducing progression of geographic atrophy in a subject, comprising:
selecting for treatment with the drug a subject having a baseline geographic atrophy lesion area of greater than or equal to 8 mm²,
administering the drug to the subject,
determining geographic atrophy lesion area, and
repeating said administering and said determining n times, where n is at least 1,
wherein said drug is effective to reduce progression of geographic atrophy if the change in lesion area determined after said repeating from baseline geographic atrophy lesion area is less than a reference subject with a baseline geographic atrophy lesion area of greater than or equal to 8 mm² treated n times with a placebo or sham treatment.

13. The method of claim 12, wherein determining comprises transforming the geographic atrophy (GA) lesion area to an effective diameter (ED) using the equation
ED=2*{square root over ((GA lesion area/π)}

14. The method of claim 13, wherein said determining step is performed at about 12 months or less after said administering.
